(19) United States
(12) Patent Application Publication
(10) Pub. No.: US 2008/0124296 A1
(43) Pub. Date: May 29, 2008

(54) COMPOSITIONS CONTAINING MICROSPHERES

(75) Inventors: Carol Elmasry, South Amboy, NJ (US); Jean-Paul Marchetti, Westfield, NJ (US); Murat Quadir, Scotch Plains, NJ (US)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL S.A., Paris (FR)

(21) Appl. No.: 11/943,849

(22) Filed: Nov. 21, 2007

Related U.S. Application Data
(60) Provisional application No. 60/866,930, filed on Nov. 22, 2006.

(51) Int. Cl. A61K 8/81; A61K 8/11; A61K 8/55

(52) U.S. Cl. 424/70.19; 424/489; 510/122

(57) ABSTRACT

The invention relates to compositions containing a microsphere, a phospholipid, an amphoteric surfactant and a nonionic surfactant, as well as to methods of applying such compositions to keratin materials and to methods of preparing such compositions.

### CROSS-REFERENCE TO RELATED APPLICATIONS

The present application claims priority from U.S. provisional patent application Ser. No. 60/866,930, filed Nov. 22, 2006, the entirety of which is hereby incorporated by reference.

### FIELD OF THE INVENTION

The present invention generally relates to cosmetic or dermatologic compositions comprising a microsphere, a phospholipid, an amphoteric surfactant and a nonionic surfactant, as well as to methods of applying such compositions to keratin materials and to methods of preparing such compositions. Such compositions possess improved properties and characteristics such as, for example, providing increased stylability, repositionability, discipline and conditioning to hair to which the compositions have been applied, even when the compositions are subsequently rinsed off.

### BACKGROUND OF THE INVENTION

Fixing keratin materials such as hair in place is an important element in styling. Such styling is particularly difficult to achieve using “rinse-out compositions,” that is compositions which are rinsed-off after application such as, for example, shampoos and/or conditioners. Typically, styling using such rinse-out compositions has been accomplished by including styling or fixing polymers in the compositions. However, the use of such polymers in rinse-out compositions has been problematic in the past, often leaving hair with less than desirable properties after application. Further improvement of such rinse-out compositions, and further improvement of the properties which such compositions provide to hair, continue to be sought.

Accordingly, one aspect of the present invention is a care and/or treatment composition for keratin materials such as hair which is able to address or overcome at least some of the problems discussed above.

### SUMMARY OF THE INVENTION

The present invention relates to cosmetic or dermatologic compositions comprising a microsphere, a phospholipid, an amphoteric surfactant and a nonionic surfactant.

The present invention relates to cosmetic or dermatologic compositions which are free of, essentially free of or substantially free of styling or fixing polymers comprising a microsphere, a phospholipid, an amphoteric surfactant and a nonionic surfactant.

The present invention further relates to cosmetic or dermatologic compositions comprising a microsphere, a phospholipid, an amphoteric surfactant and a nonionic surfactant, and further comprising a conditioning agent and/or a cleansing agent.

The present invention also relates to compositions for hair such as sprays, mousses, gels, conditioners, shampoos, conditioners, permanent waving compositions, hair care products, hair treatment products, and hair styling products comprising a microsphere, a phospholipid, an amphoteric surfactant and a nonionic surfactant.

The present invention also relates to methods of cleansing or shampooing hair comprising applying to hair a composition comprising a microsphere, a phospholipid, an amphoteric surfactant, a nonionic surfactant, and a cleansing agent, and then rinsing off the composition.

The present invention also relates to methods of conditioning hair comprising applying to the hair a composition comprising a microsphere, a phospholipid, an amphoteric surfactant, a nonionic surfactant, and a conditioning agent, and then rinsing off the composition.

The present invention also relates to methods of increasing hair body, suppleness, stylability, repositionability, combability, smoothness and/or discipline comprising applying to hair a hair body, suppleness, stylability, repositionability, combability, smoothness and/or discipline increasing effective amount of a composition comprising a microsphere, a phospholipid, an amphoteric surfactant and a nonionic surfactant.

The present invention also relates to methods of shampooing and conditioning hair comprising applying to the hair a composition comprising a microsphere, a phospholipid, an amphoteric surfactant, a nonionic surfactant, a cleansing agent and a conditioning agent, and then rinsing off the composition.

The present invention also relates to methods of holding the shape or curl of a keratin material such as hair comprising applying to the keratin material a shape or curl holding effective amount of a composition comprising a microsphere, a phospholipid, an amphoteric surfactant and a nonionic surfactant.

The present invention also relates to methods of treating or caring for keratin materials such as hair by applying compositions of the present invention to the keratin materials in an amount sufficient to treat and/or care for the keratin materials.

The present invention further relates to methods of enhancing the appearance of keratin materials such as hair by applying compositions of the present invention to the keratin materials in an amount sufficient to enhance the appearance of the keratin materials.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Cosmetically acceptable medium” means a medium that is compatible with any keratin material, such as the skin, the hair, the nails, the eyelashes, the eyebrows, the lips and any other area of body or facial skin.

“Long wear” compositions as used herein, refers to compositions where at least one property chosen from consistency, texture, and shape remains the same as at the time of application, as viewed by the naked eye, or decreases very slowly so that the naked eye could not see a difference as at the time of application, after an extended period of time, such as, for example, 1 hour, 2 hours, and further such as 8 hours. Long wear properties may be evaluated by any method known in the art for evaluating such properties. For example, long wear may be evaluated by a test involving the application of a composition to human hair and evaluating the consistency, texture and shape of the composition after an extended period of time. For example, the consistency, texture and shape of a hair composition may be evaluated immediately following application and these characteristics may then be re-evaluated and compared after an individual has worn the hair composition for a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a hair composition may be applied to hair, which may then be placed in water for a certain amount of time, such as, for example, 20 minutes. Upon expiration of the pre-ascertained amount of time, the hair may be removed from the water and passed over a material, such as, for example, a sheet of paper. The extent of residue left on the material may then be evaluated and compared with other compositions, such as, for example, commercially available compositions. For example, a composition may be waterproof if a majority of the product is left on the wearer, e.g., hair.

The term “reshapable” means to provide a hairstyle (fixability) and/or hold that can be restored or modified without additional material or heat being applied. This does not mean that additional product and/or heat may not be applied. It may be desirable to add heat and/or additional styling compositions in terms of speed, ease of use, if hair becomes unduly wet or dirty, is excessively combed, brushed or manipulated, washed, or when hair is to be dramatically restyled. At least some of the compositions in accordance with the invention should be reshapable, as judged by a professional hair stylist of ordinary skill, for at least 4 hours and up to 24 hours or more after initial application. Preferably and merely for example, in order to restore or modify the hairstyle in case of “drooping” or loss of setting (dishevelment), no new materials, such as water or any form of fixing agent, or heat are required. Other terms, which may be synonymous with reshapable, include repositionable, remoidable, restyleable, rearrangeable, and remodellable. The term “reshapable” also means to provide a hairstyle that can retain or hold a desired shape or configuration until water, heat, time and/or physical contact destroys the desired shape or configuration.

The cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to keratin materials.

The composition of the present invention may be in any form. For example, it may be a liquid, paste, a solid, gel, mousse or a cream. The composition of the invention may be transparent or clear, including for example, a composition without pigments. The composition can also be a molded composition or cast as a stick or a dish. The composition in one embodiment is a solid such as a molded stick or a poured stick. The compositions of the present invention may also be in the form of a mascara product, a hair gel, a hair mousse, or a hair spray (aerosol or non-aerosol).

As defined herein, stability is tested by placing the composition in a controlled environment chamber for 8 weeks at 25° C. In this test, the physical condition of the sample is inspected as it is placed in the chamber. The sample is then inspected again at 24 hours, 1 week, 2 weeks, 4 weeks and 8 weeks. At each inspection, the sample is examined for abnormalities in the composition such as phase separation if the composition is in the form of an emulsion. The stability is further tested by repeating the 8-week test at 37° C., 45° C., 4° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

Microsphere

According to the present invention, compositions comprising at least one microsphere are provided. Preferably, the microspheres have adhesive properties and/or are not film forming agents. Most preferably, the microspheres have adhesive properties and are not film forming agents. Suitable microspheres include but are not limited to Gel Tac microspheres (available from API: Advanced Polymers International) such as, for example, those found in Gel Tac commercial products Gel Tac 100 A, Gel Tac 100 B, Gel Tac 100 C, Gel Tac 100 K and Gel Tac 205D, as well as those microspheres disclosed in U.S. patent application publication nos. 2006/0024255 and 2002/0041858, the entire contents of both of which are hereby incorporated by reference.

The microspheres of the present invention are preferably prepared via suspension polymerization of one or more free radical polymerizable monomers in the presence of surfactants, polymerization initiators and/or suspension stabilizers. Most preferably, the microspheres are prepared by suspension polymerization of acrylic monomers in the presence of a polymerization initiator. Suitable methods of preparing the microspheres are disclosed in U.S. Pat. No. 3,691,140, U.S. Pat. No. 4,166,152, U.S. Pat. No. 4,495,318, U.S. Pat. No. 4,598,112, U.S. Pat. No. 4,786,696, U.S. Pat. No. 4,839,416, U.S. Pat. No. 4,968,562, U.S. Pat. No. 5,045,569, U.S. Pat. No. 5,571,617, U.S. Pat. No. 5,409,977, U.S. Pat. No. 5,656,705, U.S. Pat. No. 5,719,247 and/or U.S. patent application publication no. 2003/0109630, the entire contents of all of which are hereby incorporated by reference.

Preferably, monomers should be chosen to yield microspheres having a glass transition temperature (Tg) below −20° C. Such monomer choice provides the microspheres with improved adhesive properties.

According to preferred embodiments, the microspheres are prepared by homo- or co-polymerization of monomer(s) selected from the group consisting of acrylates, methacrylates, acrylic acid, methacrylic acid, acrylamides, acrylonitriles, pyrrolidones, vinyl esters, and mixtures thereof. For example, monomers which may be used to prepare the microspheres include, but are not limited to, alkyl acrylates such as butyl acrylate, ethyl acrylate, methyl acrylate, and 2-ethylhexylacrylate, alkyl methacrylates such as methyl methacrylate and butyl methacrylate, polar comonomers such as acrylic acid, methacrylic acid, hydroxyethyl acrylate, hydroxypropyl acrylate, N-tertiary octylacrylamide, acrylonitrile, acrylamide, 1-vinyl-2-pyrrolidone, sodium vinyl sulfonate, vinyl esters such as vinyl acetate, vinyl propionate, vinyl neodecanoate, vinyl stearate and vinyl pivalate. Cross-linking monomers such as, for example, butanediol diacrylate and hexanediol diacrylate can also be used. Other useful monomers and combinations thereof which can be used in the practice of the invention would be apparent to one skilled in the art.

Polymerization initiators useful in microsphere polymerization include, but are not limited to, dialkyl peroxides such as lauroyl peroxide (Laurox from Akzo Nobel), diacyl peroxides such as dibenzoyl peroxide (Lucidol A75 from Elf Atochem), peroxyesters such as t-amyl peroxypivalate (Lupersol 554-M50 from Elf Atochem), t-butyl peroctoate (Luperox 26 from Elf Atochem and Trigonox 21 from Akzo Chemicals), azo compounds such as 2,2′-azobis(isobutyronitrile) (VAZO 64) and 2,2′-azobis(2-methylbutanenitrile) (VAZO 67), and 2,2′-azobis(2,4-dimethylpentanenitrile) (VAZO 52), all from DuPont. Other useful initiators and combinations thereof which can be used in the practice of the invention would be apparent to one skilled in the art.

According to particularly preferred embodiments of the present invention, 2-ethyl hexyl acrylate (monomer) and t-butyl peroctoate (as initiator) are used to prepare the micropsheres as described in U.S. patent application publication no. 2003/0109630, the entire contents of which is hereby incorporated by reference.

Microspheres of the present invention may be of widely varied shapes such as spheres, spheroids, platelets, and irregularly shaped particles. The size of the micropshere is usually described in terms of its length and/or width. In the context of the present invention, “length” of the microsphere is intended to mean the maximum distance possible to be measured by appropriate microscopy techniques, between two opposing points of the microsphere. The “width” of the microsphere is intended to mean the minimum distance possible to be measured by appropriate microscopy techniques, between two opposing points of the microsphere.

In one embodiment, the microspheres have a ratio of length to width of greater than or equal to about 1:1.

In another embodiment, the microspheres have a mean length of about 1 to about 1000 microns, preferably from 1 to less than 30 microns, preferably from 30 to 1000 microns, preferably from 30 to 100 microns, and more preferably from about 30 to about 50 microns. The mean length of the microspheres may be measured by the following methods: optical microscopy, sieving, sedimentation, radiant diffusion, absorption, Coulter's principle, laser light diffraction, X-ray, and Sensing-zone method, which are described by Clyde Orr, “Size Measurement of Particles,” in Kirk-Othmer Encyclopedia of Chemical Technology, vol. 21, pp. 106-131 (3^{rd} ed. 1983) and by Terry Allen, “Analyse et Caracterisation, Technique de I'ingenieur” in Etudes de Structure-Granulometrie, vol. 12, pp. 1040-9 to 1040-26 (1996). In one embodiment, optical microscopy is used to determine the mean length of the microsphere.

Preferably, the microspheres are present in an amount ranging from about 0.5% to about 40% by weight of the total weight of the composition, more preferably from about 1% to about 30% of the total weight of the composition, more preferably from about 2% to about 20% of the total weight of the composition, and most preferably from about 3% to about 15%, including all ranges and subranges therebetween.

Amphoteric Surfactant

According to the present invention, compositions comprising at least one amphoteric surfactant are provided. Any amphoteric surfactant(s) can be used in accordance with the present invention, Suitable amphoteric surfactants include, but are not limited to, betaines, sultaines, hydroxysultaines, alkyl amphodiacetates, alkyl amphodipropionates, and imidazolines, including salts of these compounds. Other suitable amphoteric surfactants include other fatty acid condensates such as those formed with amino acids, proteins, and the like.

Specific examples of acceptable amphoteric surfactants include, but are not limited to, those identified in U.S. Pat. No. 6,946,124, the entire contents of which is hereby incorporated by reference, as well as to cocamphodipropionates (for example, MIRANOL C2M-SF Conc. (disodium cocamphodipropionate, available under the trade name Mackam 2CSF 40 CG from McIntyre Group LTD.), CROSULTA NE C-50 (cocamidopropyl hydroxysultaine) available from Croda, disodium wheatgermimido PEG-2 sulfosuccinate available under the trade name MACKANATE WGD from McIntyre Group Ltd, and disodium soyamphodiacetate available under the trade name MACKAM 2S from McIntyre Group Ltd.

Preferably, the amphoteric surfactant(s) is/are present in an amount ranging from about 1% to about 20% by weight of the total weight of the composition, more preferably from about 1% to about 10% of the total weight of the composition, more preferably from about 2% to about 8% of the total weight of the composition, and most preferably from about 3% to about 7%, including all ranges and subranges therebetween. Often, amphoteric surfactants are available in commercial products which also contain a solvent or carrier, meaning that the commercial products contain a “% active material” of the amphoteric surfactant. It is to be understood that the above percentages refer to such % active materials.

Nonionic Surfactant

According to the present invention, compositions comprising at least one nonionic surfactant are provided. Any nonionic surfactant(s) can be used in accordance with the present invention.

In general, nonionic surfactants having a Hydrophilic-Lipophilic Balance (HLB) of 8 or more, preferably from 8 to 20, are contemplated for use by the present invention. Nonlimiting examples of nonionic surfactants useful in the compositions of the present invention are disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C12-C50 range, preferably in the C16-C40 range, more preferably in the C24 to C40 range, and having from about 1 to about 110 alkoxy groups. The alkoxy groups are selected from the group consisting of C2-C6 oxides and their mixtures, with ethylene oxide, propylene oxide, and their mixtures being the preferred alkoxides. The alkyl chain may be linear, branched, saturated, or unsaturated. Of these alkoxylated non-ionic surfactants, the alkoxylated alcohols are preferred, and the ethoxylated alcohols and propoxylated alcohols are more preferred. The alkoxylated alcohols may be used alone or in mixtures thereof. The alkoxylated alcohols may also be used in mixtures with those alkoxylated materials disclosed herein-above. A specific example of an alkoxylated alcohol includes, but is not limited to, PPG-5-ceteth-20 (commercially available under the trade name Procetyl AWS and from Croda, Inc.)

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from Uniqema, Paterson, N.J. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, e.g. C8-30 alcohols, with sugar or starch polymers. These compounds can be represented by the formula (S)n —O—R wherein S is a sugar moiety such as glucose, fructose, mannose, galactose, and the like; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C8-20 alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein are sucrose ester surfactants such as sucrose cocoate and sucrose laurate.

Other nonionic surfactants suitable for use in the present invention are glyceryl esters and polyglyceryl esters, including but not limited to, glyceryl monoesters, preferably glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C16-C22 saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C16-C22 saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from ICI Specialty Chemicals, Wilmington, Del.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipaimitates (e.g., SPAN® 40), and sorbitan isostearate. Sorbitan monoisostearate and sorbitan sesquioleate are particularly preferred emulsifiers for use in the present invention.

Also suitable for use herein are alkoxylated derivatives of glyceryl esters, sorbitan esters, and alkyl polyglycosides, wherein the alkoxy groups is selected from the group consisting of C2-C6 oxides and their mixtures, with ethoxylated or propoxylated derivatives of these materials being the preferred. Nonlimiting examples of commercially available ethoxylated materials include TWEEN® (ethoxylated sorbitan mono-, di- and/or tri-esters of C12 to C18 fatty acids with an average degree of ethoxylation of from about 2 to about 20).

Preferred nonionic surfactants are those formed from a fatty alcohol, a fatty acid, or a glyceride with a C₄ to C₃₆ carbon chain, preferably a C₁₂ to C₁₈ carbon chain, more preferably a 16 to 18 carbon chain, derivatized to yield an HLB of at least 8. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 8. Preferably the nonionic surfactants contain ethoxylate in a molar content of from 10-25, more preferably from 10-20 moles.

Preferably, the nonionic surfactant(s) is/are present in an amount ranging from about 1% to about 40% by weight of the total weight of the composition, more preferably from about 3% to about 30% of the total weight of the composition, more preferably from about 5% to about 25% of the total weight of the composition, and most preferably from about 10% to about 20%, including all ranges and subranges therebetween. In a preferred embodiment, the amphoteric surfactant(s) and the nonionic surfactant(s) are present in a ratio of from about 1:1 to about 1:2.

Phospholipid Compound

According to the present invention, compositions comprising at least one phospholipids are provided. Most preferably, the phospholipid compound is a lecithin compound. Lecithins are mixtures of phospholipids, i.e., diglycerides of fatty acids linked to an ester of phosphoric acid. Preferably, lecithins are diglycerides of stearic, palmitic, and/or oleic acids linked to the choline ester of phosphoric acid. Lecithin is usually defined either as pure phosphatidyl cholines or as crude mixtures of phospholipids which include phosphatidyl choline, phosphatidyl serine, phosphatidyl ethanolamine, phosphatidyl inositol, other phospholipids, and a variety of other compounds such as fatty acids, triglycerides, sterols, carbohydrates, and glycolipids. Any such lecithins can be used in accordance with the present invention. The lecithin compound may be in the form of a liquid, powder, or granules.

Specific examples of acceptable types of lecithins include, but are not limited to, soy lecithin and hydroxylated lecithin. Specific lecithin compounds include, but are not limited to, ALCOLEC S which is a fluid soy lecithin, ALCOLEC F 100 which is a powder soy lecithin, and ALCOLEC Z3 which is a hydroxylated lecithin. All of these commercial products are available from the American Lecithin Company.

Also, a group of phospholipids which can be used in the present invention as phospholipids compounds include, but are not limited to, multifunctional biomimetic phospholipids, including, for example, the following multifunctional biomimetic phospholipids manufactured by Mona Industries: PHOSPHOLIPID PTC, PHOSPHOLIPID CDM, PHOSPHOLIPID SV, PHOSPHOLIPID GLA, and PHOSPHOLIPID EFA.

Preferably, the phospholipid compound(s) is/are present in an amount ranging from about 0.01% to about 10% by weight of the total weight of the composition, more preferably from about 0.1% to about 8% of the total weight of the composition, more preferably from about 1% to about 7% of the total weight of the composition, and most preferably from about 3% to about 6%, including all ranges and subranges therebetween. Since phospholipids themselves are not necessarily a pure raw material and may have free glycerides, glycerin, fatty acids, and soaps, adjustments in this amount may need to be made (e.g, one source of lecithin may require slightly different amounts to be present than other amounts to ensure that a sufficient % active material of lecithin compound is present).

When, in accordance with preferred embodiments, a lecithin compound is present, what is sometimes referred to as a “LAN system” or simply as “LAN” (Lecithin, Amphoteric, Nonionic) is present. Such LAN systems have been described in U.S. Pat. No. 6,015,574, U.S. Pat. No. 6,221,389, U.S. Pat. No. 6,524,614, U.S. Pat. No. 6,558,697, and U.S. patent application 2004/0120919, the entire contents of all of which are hereby incorporated by reference.

According to preferred embodiments, the phospholipid compound(s), the amphoteric surfactant(s), and the nonionic surfactant(s) are present such that the nonionic surfactant(s) and the amphoteric surfactant(s) are each present in an amount by weight greater than the amount of phospholipid.

According to other preferred embodiments, calculating the phospholipid as present at a value of 1, the phospholipid compound(s), amphoteric surfactant(s) and nonionic surfactant(s) are preferably present in the composition in a ratio ranging from about 1:0.75:1 to about 1:9:12, more preferably from about 1:1.5:2 to about 1:6:8. Most preferably, the ratio is about 1:3:4.

Preferably, the combined amount of phospholipid compound(s), amphoteric surfactant(s) and nonionic surfactant(s) ranges from about 1% to about 40% by weight of the total weight of the composition, more preferably from about 2% to about 25% of the total weight of the composition, more preferably from about 3% to about 20% of the total weight of the composition, and most preferably from about 4% to about 15%, including all ranges and subranges therebetween.

Additional Ingredients

The compositions of the present invention can also comprise any additive usually used in the field under consideration. For example, dispersants, antioxidants, essential oils, preserving agents, fragrances, liposoluble polymers that are dispersible in the medium, styling polymers, colorants, film forming agents, fillers, neutralizing agents, cosmetic and dermatological active agents such as, for example, emollients, moisturizers, vitamins, essential fatty acids, sunscreens, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application, including but not limited to the applications from which this application claims priority. Still further examples of such additional ingredients may be found in the International Cosmetic Ingredient Dictionary and Handbook (9^{th} ad. 2002).

According to particular aspects of the present invention, the compositions can be in the form of an emulsion. Suitable emulsion forms include but are not limited to oil-in-water, water-in-oil, oil-in-water-in-oil, water-in-oil-in-water and nanoemulsions (emulsions whose oil globules are of very fine particle size, that is to say that they have a number-average size of less than about 100 nanometers (nm)). Emulsions contain at least one oil phase and at least one aqueous phase. Typically speaking, emulsions contain surfactants or surfactant-like materials which provide stability to the emulsions and inhibit de-phasing of the emulsions.

According to particularly preferred embodiments, the composition is a “rinse-off” composition: that is, the composition is rinsed off after application. Examples of such rinse-off compositions include shampoos and conditioners.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present).

Needless to say, the composition of the invention should be cosmetically or dermatologically acceptable, i.e., it should contain a non-toxic physiologically acceptable medium and should be able to be applied to the keratin materials of human beings.

In particular, with respect to compositions for application to hair and/or eyelashes, additional optional ingredients include but are not limited to those ingredients set forth in U.S. Pat. No. 6,548,051 and U.S. patent application publication nos. 2002/0059941, 2004/0120919 and 2006/0024255, the entire contents of which are herein incorporated by reference. Examples of such additional ingredients include but are not limited to adhesive particles, non-adhesive particles, fixing (or styling) polymers, and propellants (for aerosol compositions).

However, in a particularly preferred embodiment of the present invention, a cosmetic or dermatologic composition which is free of, essentially free of or substantially free of non-adhesive particles is provided. By “free of” non-adhesive particles,” it is meant that no non-adhesive particles are present in the composition. By “essentially free of non-adhesive particles,” it is meant that less than 0.01% non-adhesive particles are present in the composition. By “substantially free of non-adhesive particles,” it is meant that less than 0.03% non-adhesive particles are present in the composition. As used herein, a “non-adhesive” particle is a particle that is not coated with an adhesive as described in U.S. Pat. No. 6,548,051, U.S. patent application publication no. 2002/0059941 and U.S. patent application publication no. 2006/0024255. More specifically, a non-adhesive particle is a discrete substrate that is not an adhesive and is not coated with an adhesive coating in whole or in part, and does not comprise an adhesive as a core or shell material when in a core/shell structure.

Similarly, in a particularly preferred embodiment of the present invention, a cosmetic or dermatologic composition which is free of, essentially free of or substantially free of styling polymers is provided. By “free of” styling polymers,” it is meant that no styling polymers are present in the composition. By “essentially free of styling polymers,” it is meant that less than 0.1% styling polymers are present in the composition. By “substantially free of styling polymers,” it is meant that less than 0.3% styling polymers are present in the composition.

Specific examples of additional ingredients include alcohols, particularly if the composition contains an aqueous phase. Any suitable alcohol may be used in accordance with the present invention such as, for example, C2-C5 alcohols like ethanol, propanol, butanol, isopropyl, etc., and mixtures thereof. If present, the alcohol is preferably present in an amount ranging from about 1% to about 80% by weight of the total weight of the composition, more preferably from about 5% to about 60% by weight, and more preferably from about 10% to about 50% by weight.

Specific examples of additional ingredients also include oils, particularly if the composition is an anhydrous composition or an emulsion. Any oils can be used in accordance with the present invention. The oils can be volatile or non-volatile, silicone-based and/or hydrocarbon-based, etc. Thus, for example, the external oil phase may contain, independently or in combination, volatile silicone oils, non-volatile silicone oils, volatile non-silicone oils and non-volatile non-silicone oils.

In one embodiment, the compositions of the present invention are substantially free of silicone oils (i.e., contain less than about 1% of silicone oil). In another embodiment, the compositions are substantially free of non-silicone oils (i.e., contain less than about 1% of non-silicone oil). In another embodiment, the compositions are substantially free of non-volatile oils (i.e., contain less than about 1% of non-volatile oil). In yet another embodiment, the compositions are substantially free of volatile oils (i.e., contain less than about 1% of volatile oil).

According to one embodiment, the oil phase may contain one or more volatile silicone oils. Examples of such volatile silicone oils include linear or cyclic silicone oils having a viscosity at room temperature less than or equal to 6 cSt and having from 2 to 7 silicone atoms, these silicones being optionally substituted with alkyl or alkoxy groups of 1 to 10 carbon atoms. Suitable oils that may be used in the invention include octamethyltetrasioxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

Further, a volatile linear silicone oil may be employed in the compositions of the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

The volatility of the solvents/oils can be determined using the evaporation speed as set forth in U.S. Pat. No. 6,338,839.

Examples of other silicone oils that may be used in the invention include non-volatile linear polydimethylsiloxanes (PDMSs), that are liquid at room temperature; polydimethylsiloxanes comprising alkyl, alkoxy or phenyl groups, which are pendent and/or at the end of a silicone chain, these groups each containing from 2 to 24 carbon atoms; phenylsilicones, for instance phenyl trimethicones, phenyl dimethicones, phenyl trimethylsiloxydiphenylsiloxanes, diphenyl dimethicones, diphenyl methyldiphenyl trisiloxanes and 2-phenylethyl trimethylsiloxysilicates.

According to other preferred embodiments, the oil phase may contain one or more non-silicone volatile oils and may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

Examples of other non-silicone oils which can be used in the compositions of the present invention include polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including and better still from 7 to 19 carbon atoms, and R₆ represents a branched hydrocarbon-based chain containing from 1 to 40 carbon atoms, including and better still from 3 to 20 carbon atoms, with R₆+R₇≧10, such as, for example, Purcellin oil (cetostearyl octanoate), isononyl isononanoate, C12 to C15 alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters,
synthetic ethers containing from 10 to 40 carbon atoms;
C₈ to C₂₆ fatty alcohols, for instance oleyl alcohol; and
mixtures thereof.

Preferably, the oils, if present, represent from about 5% to about 80% by weight of the total weight of the composition, more preferably from about 10% to about 60% of the total weight of the composition, and most preferably from about 15% to about 50%, including all ranges and subranges therebetween.

Water, when present, preferably represents from about 1% to about 90% by weight of the total weight of the composition, more preferably from about 5% to about 80% of the total weight of the composition, and most preferably from about 10% to about 70%, including all ranges and subranges therebetween.

According to the present invention, the compositions may optionally comprise at least one coloring agent. Suitable coloring agents include but are not limited to pigments (both goniochromatic and monochromatic), dyes, such as liposoluble dyes, nacreous pigments, and pearling agents. Typically, when the composition contains colorants, it is a coloring composition such as a mascara, lipstick or foundation. Alternatively, when the composition does not contain colorants, it is a clear or transparent composition which can be used as a basecoat (or topcoat) prior to (or after) application of a coloring composition to keratin materials. However, it is possible that topcoats or basecoats could contain colorants.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%.

The pigments, which may be used according to the present invention, may be chosen from white, colored, inorganic, organic, polymeric, nonpolymeric, coated and uncoated pigments. They may also be monochromatic or goniochromatic (for example, a multilayer interference structure) such as those disclosed in U.S. Pat. No. 6,451,294, the entire disclosure of which is hereby incorporated by reference. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0,5% to 40%, and further such as from 2% to 30%. In the case of certain products, the pigments, including nacreous pigments, may, for example, represent up to 50% by weight of the composition.

The compositions may also optionally comprise at least one oxidation dye chosen from oxidation bases and oxidation couplers. In one embodiment, the compositions can comprise at least one oxidation base. Suitable oxidation bases include those conventionally known as oxidation dyes, Representative oxidation dyes include ortho- and para-phenylenediamines, double bases, ortho- and para-aminophenois and heterocyclic bases as well as their addition salts with an acid such as those described in U.S. patent application publication no. 2002/0013972, the entire contents of which is hereby incorporated by reference.

Among the para-phenylenediamines, mention may be made of para-phenylenediamine, 2-methyl-para-phenylenediamine, 1-(N-ethyl-N′-β-hydroxyethyl)-amino-4-aminobenzene, 1-N,N′-bis(β-hydroxyethyl)amino-4-aminobenzene, 1-N,N′-bis(β,γ-dihydroxypropyl)amino-4-aminobenzene, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, 2-methyl-5-methoxy-para-phenylenediamine, 2,6-dimethyl-5-methoxy-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, 4-amino-N,N-diethyl-2-methylaniline, N,N-bis(β-hydroxyethyl)-para-phenylenediamine, 4-amino-N,N-bis(β-hydroxyethyl)-3-methylaniline, 4-amino-3-chloro-N,N-bis(β-hydroxyethyl)aniline, 4-amino-N-ethyl-N-carbamylmethyl-aniline, 4-amino-3-methyl-N-ethyl-N-carbamylmethyl-aniline, 4-amino-N-ethyl-N-β-piperidinoethyl)aniline, 4-amino-3-methyl-N-ethyl-N-(β-piperidinoethyl) aniline, 4-amino-N-ethyl-N-(β-morpholinoethyl)aniline, 4-amino-3-methyl-N-ethyl-N-(β-morpholinoethyl)aniline, 4-amino-ethyl-N-(β-acetylaminoethyl)aniline, 4-amino-N-(β-methoxyethyl)aniline, 4-amino-3-methyl-N-ethyl-N-(β-acetylaminoethyl)aniline, 4-amino-N-ethyl-N-(β-mesylaminoethyl)aniline, 4-amino-3-methyl-N-ethyl-N-(β-mesylaminoethyl) aniline,4-amino-N-ethyl-N-(β-sulphoethyl)aniline, 4-amino-3-methyl-N-ethyl-N-(β-sulphoethyl)aniline, N-[4′-(amino)phenyl]morpholine, N[4′-(amino)phenyl]piperidine, N-(4′-aminophenyl)-para-phenylenediamine, N-phenyl-para-phenylenediamine, 2-fluoro-para-phenylenediamine, 2-carboxy-para-phenylenediamine, 2-sulpho-para-phenylenediamine, 2-isopropyl-para-phenylenediamine, N-(β hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β, γ-dihydroxypropyl)-para-phenylenediamine, 2-β-hydroxyethyloxy-para-phenylenediamine, 2-β-acetylaminoethyloxy-para-phenylenediamine, N-β-methoxyethyl-para-phenylenediamine, para-toluoylenediamine, 2-n-propyl-para-phenylenediamine, 1,β-methoxyethylamino-4-aminobenzene, 4-aminophenyl 1-(3-hydroxy)pyrrolidone, and acid addition salts thereof.

Among the ortho-phenylenediamines, mention may be made of 4-Methyl-o-Phenylenediamine, and acid addition salts thereof. As used herein, the term double bases means compounds comprising at least two aromatic nuclei having at least one of amino and hydroxyl groups.

Among the double bases that can be used as oxidation bases in the dye compositions disclosed herein, mention may be made of amino, mono(C₁-C₄)alkylamino, di(C₁-C₄)alkylamino, tri(C1-C4)alkylamino, monohydroxy(C₁-C₄)alkylamino, imidazolinium, and ammonium radicals. Mention may also be made of N,N′-bis-(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetra-methylenediamine, N,N′-bis(β-hydroxyethyl)-N,N′-bis-(4-aminophenyl) tetramethylenediamine, N,N′-bis(4-methylaminophenyl) tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and the acid addition salts thereof.

Among the para-aminophenols, mention may be made of para-aminophenol, 4-amino-3-methylphenol, 4-amino-3-fluorophenol, 4-amino-3-hydroxymethylphenol, 4-amino-2-methylphenol, 4-amino-2-hydroxymethyl phenol, 4-amino-2-methoxymethylphenol, 4-amino-2-aminomethylphenol, 4-amino-2-(β-hydroxyethylaminomethyl)phenol, N-methyl-para-aminophenol, and the acid addition salts thereof.

The ortho-aminophenois that may be used as oxidation bases in the context of certain embodiments may be chosen from 2-aminophenol, 2-amino-1-hydroxy-5-methylbenzene, 2-amino-1-hydroxy-6-methylbenzene, 5-acetamido-2-aminophenol, and the acid addition salts thereof.

Among the heterocyclic bases that can be used as oxidation bases in the dye compositions in accordance with certain embodiments, mention may be made of pyridine derivatives, pyrimidine derivatives, pyrazole derivatives, pyrazolinone derivatives, and the acid addition salts thereof.

Among the pyridine derivatives, mention may be made of the compounds described, for example, in patents GB 1,026,978 and GB 1,153,196, as well as the compounds 2,5-diaminopyridine, 2-(4-methoxyphenyl)amino-3-aminopyridine, 2,3-diamino-6-methoxypyridine, 2-(β-methoxyethyl)amino-3-amino-6 methoxypyridine, 3,4-diaminopyridine, and the acid addition salts thereof.

Among the pyrazole and pyrazolinone derivatives, mention may be made the compounds described in patents DE 3,843,892, DE 4,133,957 and patent applications WO 94/08969, WO 94/08970, FR-A-2,733,749, and DE 195 43 988, such as 4,5-diamino-1-methyl-pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1 (β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropyl pyrazole, 4,5-diamino-3-methyl-1-isopropylpyrazole, 4-amino-5-(2′-aminoethyl)amino-1,3-dimethyl-pyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, 2-(4,5-diamino-1H-pyrazol-1-yl), and the acid addition salts thereof.

The oxidation bases, if present, may be employed in amounts ranging from 0.0001% to 12% by weight; from 0.001% to 8% by weight, all weights being based on the total weight of the composition.

The couplers that may be used in the dyeing method disclosed herein include those conventionally used in oxidation dye compositions, that is to say meta-aminophenols, meta-phenylenediamines and meta-diphenols, naphthols and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and the acid addition salts thereof.

These couplers may be chosen, for example, from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino 1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole, 6-methyl pyrazolo [1,5-a]-benzimidazole, and the acid addition salts thereof.

When they are present, these couplers may be present in amounts ranging from 0.0001% to 12% by weight; from 0.001% to 8% by weight, all weights being based on the total weight of the composition. In general, the acid addition salts of the oxidation bases and couplers may be chosen from hydrochlorides, hydrobromides, sulphates, tartrates, lactates, and acetates.

The composition disclosed herein may also comprise at least one direct dye. This at least one direct dye may be chosen from neutral, cationic, and anionic nitro dyes, azo dyes, and anthraquinone dyes, and may be present in amounts ranging from 0.001% to 20% by weight; from 0.01% to 10% by weight, all weights being based on the total weight of the composition.

According to particularly preferred embodiments of the present invention, the composition further comprises at least one conditioning agent, at least one cleansing agent, or both. Suitable conditioning agents include those which are typically found in conditioners. Suitable cleansing agents include those which are typically found in shampoos.

Specific examples of suitable cleansing agents include foaming surfactants such as, for example, anionic, non-ionic, or amphoteric surfactant materials. Suitable anionic types of surfactant include alkyl sulphates, such as lauryl, oleyl and stearyl sulphates, alkyl sulphonates, alkyl ether sulphates, alkyl glycerol ether sulphonates, alkyl phosphate esters, ethoxylated alkyl phosphate esters, sarcosinates, taurate derivatives, alkyl sulphoacetates, hydroxyalkyl sulphonate esters, such as isethionate esters, particularly of fatty carboxylic acids, for example cocoyl isethionic acid, lauryl isethionic acid and stearyl isethionic acid (usually used as the sodium salt), alkyl sulphosuccinates, such as sodium and/or potassium lauryl, oleyl and stearyl sulphosuccinates, alkyl sulphosuccinamates, and acyl glutamates. Such anionic surfactants are usually used as metal, usually alkali metal especially sodium or potassium, ammonium or ethanolamine salts. The alkyl chains in such surfactants are typically C₁₂₋₂₀, more usually C₁₄₋₁₈ chains. Suitable non-ionic types of surfactant include alkyl polysaccharides especially alkyl polyglucosides, sorbitan ester alkoxylates, and glucamide surfactants. Suitable amphoteric surfactants include betaine derivatives, imidazoline derivatives, and fatty amphoacetates, particularly coco-amphoacetates. The surfactants may also include soaps, particularly fatty acid alkali metal soaps such as sodium and/or potassium stearate. Suitable amphoteric surfactants include alkylbetaines such as dimethylbetaine, cocobetaine; alkylsulphobetaines; alkylamidopropylbetaines such as cocoamidopropylbetaine, alkylsultanes such as cocoamidopropylhydroxysultane; alkali metal or alkaline-earth metal alkylcarboxyglycinates; imidazolines; amphoacetates such as disodium cocamphodiacetate.

Specific examples of suitable conditioning agents include, but are not limited to, cationic compounds, cationic polymers, silicones and silicone derivatives, esters, hydrocarbons, alcohols, synthetic and natural oils and extracts known to be effective in conditioning hair. Suitable silicones include linear silicones (e.g., dimethicone), cyclic silicones (e.g., cyclomethicone), and alkoxylated silicones (e.g., dimethicone copolyol). Suitable esters include glyceryl esters (e.g., caprylic/capric triglyceride) and fatty acid esters (e.g., sorbitan oleates and C12-C15 benzoates). Hydrocarbons contain only carbon and hydrogen, and are generally derived from petrochemicals, but some of them are found in the plant or animal kingdom (e.g., squalene). Their structures can vary widely, and include aliphatic, alicyclic, and aromatic compounds. Typical hydrocarbons include petrolatum, paraffin, and mineral oil. Volatile hydrocarbons such as, for example, Isoeicosane, Isodocane, and Isohexadecane are also acceptable. Alcohols are organic compounds in which a hydroxyl group (—OH) is attached to a saturated carbon atom. Alcohols have the general formula ROH, where R may be aliphatic or alicyclic and may include aromatic rings. Polyols, phenols and glycols are also alcohols, and can also be used as conditioning agents.

The conditioning agent and/or cleansing agent, if present, may be present in the composition in amounts typical for conditioners and/or shampoos. Preferably, the conditioning agent, if present, is present in an amount ranging from 1% to 20% by weight, more preferably from about 3% to about 15% by weight, all weights being based on the total weight of the composition. Also preferably, the cleansing agent, if present, is present in an amount ranging from 1% to 50% by weight, more preferably from about 3% to about 30% by weight, all weights being based on the total weight of the composition.

According to preferred embodiments, methods of increasing hair body, suppleness, stylability, repositionability, combability, smoothness and/or discipline comprising applying to hair a hair body, suppleness, stylability, repositionability, combability, smoothness and/or discipline increasing effective amount of a composition comprising a microsphere, a phospholipid, an amphoteric surfactant and a nonionic surfactant are provided. The compositions may be applied to hair materials as needed, preferably once daily.

According to preferred embodiments, cleansing or shampooing hair comprising applying to hair a composition comprising a microsphere, a phospholipid, an amphoteric surfactant, a nonionic surfactant, and a cleansing agent, and then rinsing off the composition are provided. The compositions may be applied to hair materials as needed, preferably once or twice a day, more preferably once daily.

According to preferred embodiments, methods of conditioning hair comprising applying to the hair a composition comprising a microsphere, a phospholipid, an amphoteric surfactant, a nonionic surfactant, and a conditioning agent, and then rinsing off the composition are provided. The compositions may be applied to hair materials as needed, preferably once or twice a day, more preferably once daily.

According to preferred embodiments, methods of shampooing and conditioning hair comprising applying to the hair a composition comprising a microsphere, a phospholipid, an amphoteric surfactant, a nonionic surfactant, a cleansing agent and a conditioning agent, and then rinsing off the composition are provided. The compositions may be applied to hair materials as needed, preferably once or twice a day, more preferably once daily.

According to preferred embodiments, methods of holding the shape or curl of a keratin material such as hair comprising applying to the keratin material a shape or curl holding effective amount of a composition comprising a microsphere, a phospholipid, an amphoteric surfactant and a nonionic surfactant are provided.

According to yet other embodiments of the present invention, methods of styling a keratin material such as hair comprising applying to the keratin material a keratin material styling effective amount of a composition comprising a microsphere, a phospholipid, an amphoteric surfactant and a nonionic surfactant are provided.

According to yet other embodiments, methods of treating or caring for keratin materials such as hair by applying compositions of the present invention to the keratin material in an amount sufficient to treat and/or care for the keratin materials are provided.

According to other preferred embodiments, methods of enhancing the appearance of keratin materials such as hair by applying compositions of the present invention to the keratin materials in an amount sufficient to enhance the appearance of the keratin materials are provided.

In accordance with the preceding preferred embodiments, the compositions of the present invention are applied topically to keratin materials in an amount and for a time sufficient to accomplish the desired result. Thus, for example, where the compositions are rinsed off after application, the compositions are allowed to remain on the keratin material (e.g., hair) for a sufficient amount of time to allow the compositions to provide the keratin material with the desired characteristics. Preferably, the compositions are allowed to remain on the keratin material for at least 15 seconds, more preferably for at least 30 seconds, and more preferably for at least one minute before rinsing off the composition.

The present invention also envisages kits and/or prepackaged materials suitable for consumer use containing one or more compositions according to the description herein (for example, kits containing (1) shampoo; and (2) a conditioner). In a particularly preferred embodiment, a kit in accordance with the present invention comprises (1) a shampoo and/or a conditioner comprising at least one microsphere, at least one amphoteric surfactant, at least one phospholipid and at least one nonionic surfactant; and (2) a coloring composition comprising at least one coloring agent, preferably at least one oxidative dye. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLE 1

### Formulations

The following compositions were prepared.

| | Invention | Comparative |
|---|---|---|
| | Composition; | Composition |
| | Percent by | Percent by |
| | weight in the | weight in the |
| Ingredient | composition | composition |
| Lecithin | 0.1 | 0.1 |
| Acrylates/ethylhexyl acrylate | 1.5 | |
| copolymer | | |
| Polyquaternium-10 | 1.5 | 1.5 |
| Octylacrylamide/acrylates/butyl | | 1.5 |
| Aminoethyl methacrylate | | |
| copolymer | | |
| Hexylene glycol | 1.0 | 1.0 |
| Cocamidopropyl betaine | 1.75 | 1.75 |
| PPG-5 Ceteth-20 | 3.1 | 3.1 |
| Sodium laureth sulfate | 9.7 | 9.7 |
| Disodium | 1.8 | 1.8 |
| cocoamphodipropionate | | |
| Cosmetic additives, fragrance, | 0.85 | 0.85 |
| preservatives | | |
| Q.S. water | | |

The above compositions were then subjected to various testing. The results of the testing set forth below showed that the invention composition possessed demonstrably better smooth hair feel, wet hair combing, wet hair suppleness, wet hair Smoothness, body, dry hair suppleness, dry hair combing discipline, and repositionability properties than the comparative composition.

What is claimed is:

1. A rinse-off composition comprising at least one adhesive microsphere, at least one amphoteric surfactant, at least one phospholipid and at least one nonionic surfactant.

2. The rinse-off composition according to claim 1, wherein the phospholipid compound(s), the amphoteric surfactant(s) and the nonionic surfactant(s) are present in a ratio of from about 1:1.5:2 to about 1:6:8.

3. The rinse-off composition according to claim 2, wherein the phospholipid compound(s), the amphoteric surfactant(s) and the nonionic surfactant(s) are present in a ratio of about 1:3:4.

4. The rinse-off composition according to claim 2, wherein the combined amount of phospholipid compound(s), amphoteric surfactant(s) and nonionic surfactant(s) ranges from about 3% to about 20% by weight with respect to the total weight of the composition.

5. The rinse-off composition according to claim 4, wherein the combined amount of phospholipids compound(s), amphoteric surfactant(s) and nonionic surfactant(s) ranges from about 4% to about 15% by weight with respect to the total weight of the composition.

6. The rinse-off composition of claim 1, wherein the adhesive microsphere is prepared by polymerization of monomer(s) selected from the group consisting of acrylates, methacrylates, acrylic acid, methacrylic acid, acrylamides, acrylonitriles, pyrrolidones, vinyl esters, and mixtures thereof.

7. The rinse-off composition of claim 1, wherein the adhesive microsphere is prepared by polymerization of monomer(s) selected from the group consisting of acrylates, methacrylates, acrylic acid, methacrylic acid, and mixtures thereof.

8. The rinse-off composition of claim 1, wherein the adhesive microsphere is prepared by polymerization of ethylhexylacrylate.

9. The rinse-off composition of claim 1, wherein the adhesive microsphere has a mean length of about 30 to about 1000 microns.

10. The rinse-off composition of claim 1 wherein the adhesive microsphere has a mean length of about 30 to about 100 microns.

11. The rinse-off composition of claim 1, further comprising at least one conditioning agent.

12. The rinse-off composition of claim 1, further comprising at least one cleansing agent.

13. The rinse-off composition of claim 11, further comprising at least one cleansing agent.

14. A method of increasing hair body, suppleness, stylability, repositionability, combability, smoothness and/or discipline comprising applying to hair a hair body, suppleness, stylability, repositionability, combability, smoothness and/or discipline increasing effective amount of a composition comprising at least one adhesive microsphere, at least one phospholipid, at least one amphoteric surfactant and at least one nonionic surfactant.

15. A method of cleansing or shampooing hair comprising applying to hair a composition comprising at least one adhesive microsphere, at least one phospholipid, at least one amphoteric surfactant and at least one nonionic surfactant.

16. A method of conditioning hair comprising applying to the hair a composition comprising at least one adhesive microsphere, at least one phospholipid, at least one amphoteric surfactant and at least one nonionic surfactant.
